(19) United States
(12) Patent Application Publication
(10) Pub. No.: US 2003/0229011 A1
(43) Pub. Date: Dec. 11, 2003

(54) Humanized lactoferrin and uses thereof

(76) Inventors: Steven O. Braun, Oneonta, NY (US); Nico Van Belzen, Veghel (NL); Ram Nimmagudda, Oneonta, NY (US)

Correspondence Address: NIXON & VANDERHYE, PC, 1100 N GLEBE ROAD, 8TH FLOOR, ARLINGTON, VA, 22201-4714, US

(21) Appl. No.: 10/326,269

(22) Filed: Dec. 23, 2002

Related U.S. Application Data
(60) Provisional application No. 60/342,747, filed on Dec. 28, 2001.

(51) Int. Cl. A61K 38/40

(52) U.S. Cl. 514/6

(57) ABSTRACT

This invention relates, generally, to lactoferrin and, more specifically, to immobilized humanized lactoferrin and uses thereof.

This application claims priority from Provisional Appln. 60/342,747, filed Dec. 28, 2001, the entire content of which is incorporated herein by reference.

### TECHNICAL FIELD

This invention relates, generally, to lactoferrin and, more specifically, to immobilized humanized lactoferrin and to the use thereof in effecting reduction of microbial contamination while avoiding the risk of CMA.

### BACKGROUND

Lactoferrin, an iron binding glycoprotein, is present in various secretions of mammals. It plays an important role in iron transport and utilization, cell-mediated host immunity, and neutralizes pathogenic microorganisms by preventing them from obtaining necessary iron at the site of entry, thereby preventing the spread of infection (sequester iron).

Milk is a good source of lactoferrin. However, the severely limited amount of human milk restricts human lactoferrin production. Furthermore, production of lactoferrin from human milk presents a risk factor of infectious contamination. That is, it can be associated with a potentially lethal contaminant, such as the human immunodeficiency virus (HIV) or another undesirable agent.

Recombinantly produced human lactoferrin provides an alternative to lactoferrin isolated from natural human sources, as does bovine milk-derived lactoferrin. Since human and bovine lactoferrins share a 70% homology in primary amino acid structure, and considerable homology in three dimensional structure, as well as in function, bovine milk-derived lactoferrin offers the same biological benefits as human lactoferrin.

Although bovine milk and lactoferrin are well-tolerated and beneficial to the large majority of the population, a small but significant subgroup of humans suffers from cow's milk allergy (CMA). CMA in humans is well documented. The major cow's milk allergens in IgE-mediated cow's milk allergy are casein, β-lactoglobulin, and α-lactalbumin (Hefle et al, CRC Crit. Revs. Food Sci.-Nutr. 36:S69-S89 (1996); Wal, Int. Dairy J. 8:413-423 (1998)). Available data indicate that bovine lactoferrin is also a cow's milk allergen (Atkinson, Toxicology 91:281-288 (1994); Miller et al, Allergy 53:35-37 (1998)). It is also known that some cow's milk-allergic individuals have IgE antibodies directed against lactoferrin (Baldo, Aust. J. Dairy Technol. 39:120-128 (1984); Host et al, Allergy 47:218-229 (1992); Wal et al, Food Agric. Immunol. 7:175-187 (1995); Wal, Int. Dairy J. 8:413-423 (1998)).

In summary, in sensitive individuals, the administration of bovine lactoferrin increases the risk of CMA (allergy against bovine lactoferrin). CMA has a wide spectrum of clinical symptoms ranging from intestinal discomfort to life-threatening anaphylactic shock.

People suffering from CMA have to avoid the use of milk and milk-derived proteins. Immobilized native bovine milk-derived lactoferrin (IMDL)-treated meat contains bovine lactoferrin and thus poses a risk to some people that suffer from CMA.

The immobilization of lactoferrin to increase its antimicrobial effect, and uses thereof, are taught in U.S. Pat. No. 6,172,040. This patent, however, does not acknowledge the risk of CMA associated with the use of bovine lactoferrin. Although U.S. Pat. No. 6,172,040 lists various types of lactoferrin, data are shown for bovine lactoferrin only. Moreover, the patent does not teach the specific use of immobilized, humanized lactoferrin with the aim of safeguarding people against CMA. These disadvantages are overcome in the present invention which is based on the use of immobilized recombinant humanized lactoferrin. Because humanized lactoferrin is essentially identical to human lactoferrin, it is not recognized by the body as a foreign substance, therefore, no immune response is generated. Consequently, CMA does not occur. U.S. application Ser. No. 20020160941 (Kruzel) describes a method of reducing microbial contamination using recombinant human lactoferrin but makes no reference to increasing efficacy through immobilization and makes no reference to the use of recombinant human lactoferrin in overcoming CMA.

### SUMMARY OF THE INVENTION

This invention relates, generally, to lactoferrin and, more specifically, to immobilized humanized lactoferrin and to the use thereof in effecting reduction of microbial contamination while avoiding the risk of CMA.

Objects and advantages of the present invention will be clear from the description that follows.

### DETAILED DESCRIPTION OF THE INVENTION

The present invention relates to immobilized recombinant humanized lactoferrin (IRHL) and to methods of using same in the reduction of microbial contamination of substances, including tissues. The use of IRHL avoids the risk of allergic responses associated with native bovine milk-derived lactoferrin (NMDL) or IMDL. For the purpose of this disclosure, recombinant humanized lactoferrin is defined as a form of lactoferrin that has an amino acid sequence that is more than 90%, preferably more than 95%, more preferably more than 99%, homologous to human lactoferrin (as determined using BLAST). The preferred form of recombinant humanized lactoferrin is recombinant human lactoferrin, but lactoferrins from other mammals (like apes) can also be used. Likewise, lactoferrins produced by modification of the amino acid sequence or the nucleic acid sequence encoding lactoferrin can be used. The recombinant lactoferrin can be produced using methods such as described in U.S. Pat. No. 6,066,469 (see also U.S. Pat Nos. 5,571,591, 5,571,697, and 5,571,896). Immobilization can be effected using methods such as those described in U.S. Pat. No. 6,172,040.

IRHL can be applied to human tissues, including oral mucosoidal lining tissue, gastrointestinal epithelial lining tissue, or skin epidermal lining tissue or the collagen tissues. The IRHL can aid in growth inhibition and or microbial detachment of the microbes in human tissue applications and thus increases safety from the risks associated from usage of NMDL and IMDL. Specific embodiments include oral care formulations, wound care formulations (both external and internal wounds) (such as bandaids), and formulations to maintain healthy gastrointestinal tract. The IRHL can be present, for example, in solution (for example, as a solution suitable for administering as a spray or wash), a gel, cream or ointment. The present invention includes the use of IRHL with medicines and drugs taken into the body of a human or other animal, or applied topically.

IRHL can also be used in various stages of food processing (see, for example, U.S. Pat. No. 6,172,040). Indeed, IRHL is useful with any product prone to microbial contamination or proliferation. Representative products include processed and unprocessed foodstuffs for human or for animal consumption. IRHL is especially useful in treating whole muscle and ground meat products, including beef products, pork products and poultry products, such as sausages, salamis, hotdogs and the like. In addition, IRHL is useful in treating processed deli meats such as sliced chicken, ham, pork, turkey and the like.

IRHL is effective in treating a wide variety of microbes including bacteria, fungi, protozoa and viruses. It is especially useful in treating food-borne pathogens, food-borne radiation-resistant bacteria, and food spoilage microorganisms. Representative bacteria that can be controlled by the inventive method include: enterotoxigenic Escherichia coli, enteropathogenic Escherichia coli, Shigella dysenteriae, Shigella flexneri, Salmonella typhimurium, Salmonella abony, Salmonella dublin, Salmonella hartford, Salmonella kentucky, Salmonella panama, Salmonella pullorum, Salmonella rostock, Salmonella thompson, Salmonella virschow, Campylobacter jejuni, Aeromonas hydrophila, Staphylococcus aureus, Staphylococcus hyicus, Staphylococcus epidermidis, Staphylococcus hominis, Staphylococcus warneri Staphylococcus xylosus, Staphylococcus chromogenes, Bacillus cereus, Bacillus subtilis, Candida albicans, Brochothrix thermospacta, Bacillus pumilus, Enterococcus faecium, Deinococcus radiopugnans, Deinococcus radiodurans, Deinobacter grandis, Acinetobacter radioresistens, or Methylobacterium radiotolerans, Streptococcus mutans, Streptococcus pyogenes, Actinobacillus haemophilus, Pseudomonas aeruginosa, Porphyromonoas gingivalis, Actinobacillus actinomycetemcomitans, Prevotella intermedia, Prevotella melaninogenica, Enterobacter clocae, Proteus vulgaris, Klebsiella pneumoniae, Streptococcus faecalis, methycillin resistant staphylococcus aureus.

IRHL can be applied by any suitable method. Representative methods include spraying the product or washing during various processing steps, or coating by electrostatic spray dispersion, with an aqueous suspension or solution or dehydrated powder form containing IRHL.

The concentration of IRHL used on the surface of the tissue can range from about 0.0001 to about 100 mg/sq.inch, preferably, about 0.001 to about 10 mg/sq. inch.

### EXAMPLE I Experimental Details

Bacterial Growth-Inhibition Assay:

i) a pure colony of bacteria is picked from a tryptic soy agar (TSA) plate, inoculated in 10-ml of tryptic soy broth (TSB) and incubated at 37° C., overnight;

ii) from this overnight culture, 50 μl of TSB-grown cells is transferred to 10-ml of fresh TSB and incubated at 37° C. for 4-h (in order to get metabolically-active bacteria in log-phase of growth);

iii) bacteria are harvested by centrifugation for 3-4 min. at 4000 rpm and the pellet resuspended in citrate bicarbonate (CB) buffer, pH 8.1;

iv) the bacterial suspension is adjusted to about 1.0 O.D. (about 8-logs) at 600 nm and appropriate serial dilutions prepared to bring the cell density to 3-logs and/or 4-logs;

v) a microplate template is designed to accommodate Sterility Controls, Growth Control, and Experimental Units (the total volume of each well does not exceed 200 μl)—the protocol is always set as quadruplicates or octaplicates;

vi) Sterility Controls consist of 100 μl of double strength TSB and 100 μl of CB buffer or lactoferrin preparations (in CB buffer);

vii) Growth Control consists of 100 μl of double strength TSB, 50 μl of CB buffer, and 50 μl of bacterial suspension (3-log or 4-log cell density);

viii) Experimental Units consist of 100 μl double strength TSB, 50 μl of lactoferrin test sample (appropriate dilution) and 50 μl of bacterial suspension;

ix) 0-hour reading of the wells is measured at 420-580 nm on a microplate reader prior to incubation at 37° C. (the 0-hour reading provides background O.D. for the constituents in the well—this background reading is subtracted from the corresponding wells to make final test readings (air bubbles in the wells are avoided as they otherwise might contribute to inaccurate O.D. readings));

x) O.D. measurements are read at time points of 12-h, 18-h, and 24-h (if necessary at 48-h) (alternatively, plate counts can be taken to determine growth inhibition); and

xi) average of quadruplicate or octaplicate O.D. readings for each Experimental Unit is taken and compared with the Growth Control—percent growth is expressed as: Experimental Unit/Growth Control×100.

Bacterial Collagen Adhesion Assay:

Preparation of ³H-Thymidine Labeled Bacteria

i) a 50 μl inoculum of an overnight culture of a bacterial strain grown in trypticase soy broth (Difco) at 37° C. is re-inoculated in 10-milliliter of the same broth containing ³H-thymidine (20 μCi; ICN Laboratories);

ii) bacteria are incubated at 37° C. until the cells reach an exponential growth-phase (about 4- to 5-h) to allow optimum uptake and incorporation of ³H-thymidine into the bacteria DNA;

iii) ³H-thymidine labeled bacteria are harvested by centrifugation at 7,500×g, washed and resuspended in phosphate buffered saline (PBS, pH 7.2);

iv) correlation (r=0.98) is made between the degree of ³H-thymidine labeling (scintillation counts measured as disintegration per minute; DPM), bacterial viability (measured as total viable plate counts) and total cell counts (OD measurement at 600 nm); and

v) a final density of bacterial suspension is optically adjusted to 0.04 OD at 600 nm (corresponding to ˜10⁷ cells/milliliter).

Bacteria-Biomatrix Attachment Assay.

i) Biocoat® Cell Environments™ (Becton Dickinson, Bedford, Mass.) 24-well plates containing collagen (Cn) type-I are used in the attachment studies;

ii) matrix components are applied as an even, optically clear coating covering a total surface area of 1.75 cm²;

iii) a 2-milliliter volume of ³H-thymidine labeled bacterial suspension (2×10⁷ bacteria) is added to each well containing matrix layer and incubated for 2-h at room temperature;

iv) bacterial suspension is aspirated from the wells and discarded—one milliliter of trypsin type 1 (110 enzyme units; Sigma) is added to each well to hydrolyze for 30 min at room temperature to release the matrix protein layer;

v) the trypsin hydrolysate is aspirated into a scintillation vial, the well is further treated with 1-milliliter of tissue homogenizer (Scintigest™; Fisher Scientific) for 10 min at room temperature and the homogenate is aspirated into the corresponding vial;

vi) a volume of 10-milliliter scintillation cocktail (ScintiSafe™ Gel) is dispensed into the vial and thoroughly mixed; and

vii) after settling and clarification of the mixture, the radioactivity is measured using a liquid scintillation analyzer (Tri-Carb 2100 TR®, Packard Inc.).

Assay for Measuring Detachment of Biomatrix-Adherent Bacteria.

i) The interaction of bacteria with Cn type-I matrix is performed as described above;

ii) a 2-milliliter volume of radio-labeled bacterial suspension (2×10⁷ bacteria/milliliter) is added to each well and incubated for 2-h at room temperature;

iii) unbound bacteria are aspirated from the well;

iv) a 2 milliliter volume of lactoferrin (LF) suspension is added to each well to compete for binding-displacement of Cn-adherent radio-labeled bacteria at room temperature for 1-h;

v) the LF suspension is aspirated and the radioactivity of adherent bacteria is measured as described above;

vi) control wells of adherent bacteria without LF treatment are considered as zero interference (100% adherence).

### Results

An antimicrobial growth inhibition assay was performed using the different lactoferrins to demonstrate their inhibitory effects on the growth of E. coli serotype O157:H7 (strain ATCC43895).

Bacteria were inoculated at 2×10³ in a volume of 200 microliter and grown for 8 hours in TSB at 37° C. The number of bacteria was determined by plate counting. The data are presented in Table 1.

TABLE 1
antimicrobial activity of different lactoferrins on meat pathogen
E. coli O157:H7

| treatment | plate count | log reduction |
|---|---|---|
| no LF | 1.0 × 10⁸ | |
| NMDL | 9.0 × 10⁶ | 1.0 |
| IMDL | 8.2 × 10⁴ | 3.1 |
| NRHL | 9.2 × 10⁶ | 1.0 |
| IRHL | 9.1 × 10⁴ | 3.0 |

NMDL: native milk-derived bovine lactoferrin
IMDL: immobilized milk-derived bovine lactoferrin
NRHL: native recombinant human lactoferrin
IRHL: immobilized recombinant human lactoferrin

Antimicrobial effect of the diffent lactoferrins on wound pathogens was studied by a similar method but using a turbidometric assay as end point (Table 2).

TABLE 2
antimicrobial activity of different lactoferrins on wound pathogens

| | Growth inhibition compared to | | | |
|---|---|---|---|---|
| | control (%) | | | |
| | treatment | | | |
| Pathogen | NMDL | IMDL | NRHL | IRHL |
| Proteus vulgaris ATCC13315 | 71 | 100 | 75 | 100 |
| Pseudomonas aeruginosa | 82 | 100 | 79 | 100 |
| ATCC27853 | | | | |
| Salmonella typhimurium | 42 | 62 | 45 | 64 |
| ATCC14028 | | | | |
| Klebsiella pneumoniae ATCC13883 | 35 | 100 | 33 | 95 |
| Staphylococcus aureus ATCC25923 | 3 | 96 | 1 | 91 |
| Staphylococcus epidermidis | 61 | 94 | 55 | 92 |
| ATCC12228 | | | | |
| Streptococcus pyogenes ATCC19615 | 59 | 51 | 49 | 52 |

NMDL: native milk-derived bovine lactoferrin
IMDL: immobilized milk-derived bovine lactoferrin
NRHL: native recombinant human lactoferrin
IRHL: immobilized recombinant human lactoferrin

Antimicrobial activity of the different lactoferrins on oral pathogens was studied by inhibition of adhesion to collagen matrix or hydroxyapatite. Lactoferrins were coated to the biological surface prior to bacterial challenge (Table 3).

TABLE 3
inhibition of oral pathogen adhesion by different lactoferrins

| | Adhesion inhibition compared to | | | |
|---|---|---|---|---|
| | control (log reduction) | | | |
| | treatment | | | |
| Pathogen | NMDL | IMDL | NRHL | IRHL |
| Actinobacillus | 1 | 5 | 1 | 4 |
| actinomycetemcomitans | | | | |
| Candida albicans | 1 | 3 | 1 | 3 |
| Pseudomonas aeruginosa | 1 | 4 | 1 | 4 |
| Streptococcus mutans | 1 | 4 | 1 | 4 |

NMDL: native milk-derived bovine lactoferrin
IMDL: immobilized milk-derived bovine lactoferrin
NRHL: native recombinant human lactoferrin
IRHL: immobilized recombinant human lactoferrin

From Tables 1-3, it is concluded that:

i) immobilization increases the antimicrobial activity of lactoferrin, and

ii) immobilized bovine and recombinant human lactoferrin have similar antimicrobial activity.

### EXAMPLE II Experimental Details

A comparison of the allergenicity of bovine and recombinant human lactoferrin was carried out as follows (see also van Beresteijn et al, J. Allergy Clin. Immunol. 96:365-74 (1995)):

i) 96-well plates are coated overnight at 4° C. with 2 μg/well protein (NMDL, IMDL, NRHL, or IRHL (see Table 4) in sodium bicarbonate buffer (pH=9.6)—wells without protein coating serve as blank;

ii) after washing the plates, residual free binding sites are blocked with 135 μl 0.8% fish gelatin in PBS-Tween-20;

iii) after washing, plates are incubated with 100 μl pooled CMA (cow milk allergy) patient serum in appropriate dilutions (1000-, 2000-, and 4000-fold), or pooled negative control serum in the same dilutions;

iv) after washing, the IgE and IgG antibodies that react with the plates are determined by 100 μl of either peroxidase-conjugated goat-anti-human IgE or peroxidase-conjugated goat-anti-human IgG antibodies in PBS-Tween-20+0,8% fish gelatin;

v) after washing, a freshly prepared enzyme substrate (o-phenylenediamine dihydrochloride in citrate and potassium phosphate buffer, containing 0,012% hydrogen peroxide) is added to the wells;

vi) the plates are incubated until a sufficient color reaction has developed or to a maximum of 30 min—the reaction is terminated by adding 50 μl 4N H₂SO₄; and

vii) optical densities are read at 490 nm using an automated ELISA plate reader.

### Results

The results are provided in Table 4:

TABLE 4
allergenicity of different lactoferrins

| | treatment | | | | | | |
|---|---|---|---|---|---|---|---|
| | OD 490 nm | | | OD 490 nm | | | |
| serum | CMA patients | | | healthy individuals | | | no |
| dilution | 1000 | 2000 | 4000 | 1000 | 2000 | 4000 | serum |

IgG values

| | | | | | | | |
|---|---|---|---|---|---|---|---|
| NMDL | 0.906 | 0.521 | 0.311 | 0.122 | 0.095 | 0.113 | 0.086 |
| IMDL | 0.776 | 0.448 | 0.268 | 0.109 | 0.104 | 0.090 | 0.097 |
| NRHL | 0.455 | 0.275 | 0.196 | 0.161 | 0.127 | 0.124 | 0.099 |
| IRHL | 0.463 | 0.249 | 0.191 | 0.170 | 0.123 | 0.119 | 0.097 |
| blank | 0.08 | 0.056 | 0.051 | 0.081 | 0.051 | 0.06 | 0.062 |

IgE values

| | | | | | | | |
|---|---|---|---|---|---|---|---|
| NMDL | 0.830 | 0.566 | 0.383 | 0.174 | 0.157 | 0.153 | 0.134 |
| IMDL | 0.779 | 0.524 | 0.377 | 0.220 | 0.205 | 0.186 | 0.148 |
| NRHL | 0.433 | 0.301 | 0.274 | 0.262 | 0.231 | 0.219 | 0.187 |
| IRHL | 0.436 | 0.278 | 0.235 | 0.249 | 0.217 | 0.198 | 0.171 |
| blank | 0.101 | 0.070 | 0.089 | 0.095 | 0.135 | 0.090 | 0.077 |

NMDL: native milk-derived bovine lactoferrin
IMDL: immobilized milk-derived bovine lactoferrin
NRHL: native recombinant human lactoferrin
IRHL: immobilized recombinant human lactoferrin

From Table 4 it is concluded that:

i) bovine lactoferrin is more allergenic than recombinant human lactoferrin; and

ii) immobilization does not influence allergenicity.

All documents cited above are hereby incorporated in their entirety by reference.

What is claimed is:

1. A method of reducing microbial contamination on a surface comprising treating said surface with an amount of immobilized recombinant humanized lactoferrin (IRHL) sufficient to reduce the contamination, without introducing the risk of an allergic response associated with native bovine milk-derived lactoferrin (NMDL) or immobilized native bovine milk-derived lactoferrin (IMDL).

2. The method according to claim 1 wherein said recombinant humanized lactoferrin is a form of lactoferrin that has an amino acid sequence that is more than 90% homologous to human lactoferrin.

3. The method in accordance with claim 1, wherein the surface is an animal tissue that is suitable for consumption by a human.

4. The method in accordance to claim 1, wherein the surface is a human tissue surface.

5. The method according to claim 1, wherein the microbe is enterotoxigenic Escherichia coli, enteropathogenic Escherichia coli, Shigella dysenteriae, Shigella flexneri, Salmonella typhimurium, Salmonella abony, Salmonella dublin, Salmonella hartford, Salmonella kentucky, Salmonella panama, Salmonella pullorum, Salmonella rostock, Salmonella thompson, Salmonella virschow, Campylobacter jejuni, Aeromonas hydrophila, Staphylococcus aureus, Staphylococcus hyicus, Staphylococcus epidermidis, Staphylococcus hominis, Staphylococcus warneri Staphylococcus xylosus, Staphylococcus chromogenes, Bacillus cereus, Bacillus subtilis, Candida albicans, Brochothrix thermospacta, Bacillus pumilus, Enterococcus faecium, Deinococcus radiopugnans, Deinococcus radiodurans, Deinobacter grandis, Acinetobacter radioresistens, Methylobacterium radiotolerans, Streptococcus mutans, Streptococcus pyogenes, Actinobacillus haemophilus, Pseudomonas aeruginosa, Porphyromonoas gingivalis, Actinobacillus actinomycetemcomitans, Prevotella intermedia, Prevotella melaninogenica, Enterobacter clocae, Proteus vulgaris, Klebsiella pneumoniae, Streptococcus faecalis, or methycillin resistant Staphylococcus aureus.

6. The method in accordance with claim 3, wherein the concentration of IRHL on the surface of the animal tissue is from about 0.0001 to about 100 mg/sq.inch.

7. The method in accordance with claim 3, wherein the animal tissue is a beef product, a pork product, or a poultry product.

8. The method according to claim 7, wherein the IRHL inhibits microbial growth or enhances detachment of the microbes from the animal tissue and thereby increases the safety of the meat product without introducing risks related to cow's milk allergy that result from usage of NMDL and IMDL.

9. The method according to claim 7, wherein the animal tissue is a carcasses, ready case meat, sub primal cuts, and/or fresh processed cuts of meat.

10. The method according to claim 4, wherein the concentration of IRHL on the surface of the human tissue is from about 0.0001 to about 100 mg/sq.inch.

11. The method according to claim 4, wherein the human tissues are preferably oral mucosoidal lining tissue, gastrointestinal epithelial lining tissue, or skin epidermal lining tissue or collagen tissues.

12. The method according to claims 11, wherein the IRHL inhibits microbial growth or enhances detachment of the microbes from human tissue and thereby increases safety without introducing risks related to cow's milk allergy that result from usage of NMDL and IMDL.

13. The method according to claim 12, wherein the human tissue is oral tissue.

14. The method according to claim 12, wherein the human tissue is a wounded tissue.

15. The method according to claim 12, wherein the human tissue is gastrointestinal tissue.
